(19) United States
(12) Patent Application Publication
(10) Pub. No.: US 2002/0081745 A1
(43) Pub. Date: Jun. 27, 2002

(54) Assessment of psychiatric and neurological conditions

(76) Inventors: Marion Ann Ross, Nairn (GB); Alezander Iain Munro Glen, Kingussie (GB)

Correspondence Address: Drinker, Biddle & Reath LLP, One Logan Square, 18th and Cherry Streets, Philadelphia, PA, 19103-6996, US

(21) Appl. No.: 09/977,475

(22) Filed: Oct. 15, 2001

Related U.S. Application Data
(63) Continuation-in-part of application No. PCT/GB00/01447, filed on Apr. 14, 2000.

(30) Foreign Application Priority Data
Apr. 14, 1999 (GB) 9908485.7
Apr. 14, 1999 (GB) 9908483.2

(51) Int. Cl. G01N 33/00

(52) U.S. Cl. 436/141

(57) ABSTRACT

A method of assessing a psychiatric or neurological condition such as schizophrenia comprises measuring the presence and optionally the amount of butane (and optionally ethane) in the expired breath of the subject. In certain preferred embodiments the variation in the levels of butane is used as an indicator of the presence and optionally the severity of the condition.

### FIELD OF THE INVENTION

This invention relates to the assessment of psychiatric and neurological conditions, and particularly to identifying in non-invasive ways whether a patient is suffering from a psychiatric condition such as schizophrenia.

### BACKGROUND TO THE INVENTION

The assessment of schizophrenic patients has traditionally been conducted by observing the patient and assessing the presence and/or severity of schizophrenia by reference to behavioural markers. However, this requires skilful observation and is difficult to quantify. Kovaleva et al (Zh Neuropatol Psikiatr 1989;89:99-110 and Phillips et al (J Clin Pathol 1993;46:861-864) have measured expired pentane in the breath of suspected schizophrenics and used this as an indicator of the disease.

### SUMMARY OF THE INVENTION

According to the present invention there is provided a method for the assessment of psychiatric or neurological conditions, the method comprising determining the presence and/or amount of butane in the expired breath of the patient.

The invention also provides a method for the assessment of psychiatric or neurological conditions, the method comprising determining the presence and/or amount of ethane in the expired breath of the patient.

Preferably the concentration of butane or ethane in expired breath is measured and can be used in accordance with the invention to determine the severity of the psychiatric condition in a quantitative manner. However, simpler embodiments of the invention can be used qualitatively to determine the presence or absence of the condition in the patient.

In preferred embodiments of the invention, the condition assessed is schizophrenia, although the method may also be appropriate to other psychiatric conditions such as bipolar disorder, alcoholism, and depression, and neurological conditions such as dementia, dyslexia, Huntingdon's chorea, Parkinson's disease, dementia, Down's syndrome, Alzheimer's disease, head injury and stroke, and neurodevelopmental disorders such as attention deficit hyperactivity disorder, dyspraxia and autistic spectrum disorder.

Typically, the method determines the presence and/or concentration of butane or ethane in breath expired from the patient i.e. so that the expired breath is not returned to the patient.

The butane or ethane in expired breath is preferably thermally desorbed, can be separated by gas chromatography and can be detected and/or measured by mass spectroscopy. The expired breath to be assayed in the method is preferably captured in a breath capture syringe, and thereafter injected into an automatic thermal desorption (ATD) tube containing an absorbent matrix e.g. absorption granules such as Carbotrap 300. ATD tubes from Perkin-Elmer (part number N930-7000), Supelco (part number 25050) and Markes International Limited are suitable.

Captured breath samples in ATD tubes are preferably analysed in a gas chromatography system such as a Perkin-Elmer Autosystem XL (part number N6119101) with mass spectrometry (we used a Turbo Mass - part number N611000A) and automatic thermal desorption (ATD 400 - part number E6419001).

The ATD tube allows automatic thermal desorption of all the volatiles in the expired air injected into the tube on to a cold trap. The cold trap is then very rapidly heated up, and all volatiles are injected straight onto a gas chromatography (GC) column which separates all the volatiles into their individual components. The various components are then identified by the mass spectrometer, and the compounds of interest can then be quantified. Other ways of determining the butane or ethane present are also suitable, for example, gas chromatography, or other chromatography techniques, such as supercritical chromatography, combined with any suitable detection method such as flame ionisation detection. Various methods of adsorption and e.g. thermal desorption can be used to trap and release the volatiles onto the chromatography system used. Spectroscopy is also a useful method of detection, e.g. Infra-red chromatography or fourier transformed infra-red spectroscopy (FTIR) and laser spectroscopy. Nuclear magnetic spectroscopy (NMR)can also be used for carbon or proton spectroscopy, optionally using deuterium labelling. Differential thermal analysis (DTA) or differential scanning calorimetry (DSC) can be used to measure endothermic or exothermic effects corresponding to e.g. decomposition or chemisorption and can be used to detect butane or ethane.

U.S. Pat. Nos. 5,150,603 and 4,535,315 also disclose suitable alkane gas sensors that can be employed. Electronic devices that can detect alkanes are also widely available such as the electronic nose described by Maricou et al in Water, air and soil pollution 1998;107,423-442, Brudzewski in Sensors and actuators B.1999 55;38-46, Sommer et al in Actuators B 1992 B6(1-3), 262-5, and an optical method is shown by Guiliani et al in Actuators (1984) 6(2),107-12. All of the devices and methods cited above are incorporated herein by reference.

### DESCRIPTION OF PREFERRED EMBODIMENTS

Embodiments of the present invention will now be described by way of example and with reference to the following examples.

### EXAMPLE 1

### Butane Detection

Expired air samples were collected from patients using a syringe of approximate volume 125 mls with a piston movable along the barrel, a plunger attached to the piston and extending out from an open end of the syringe and a one-way valve at the other end of the syringe to allow influx of expired air from the patient but preventing escape of the captured breath sample. A mouthpiece was attached to the one-way valve. The patient was instructed to exhale in one long breath into the syringe until they could no longer breathe out any more, to collect the end expired air from the lungs. In other procedures, the patient's breath was collected using standard techniques optionally after the patient had been allowed to acclimatize his breathing pattern with a metronome at around ten breaths per minute so that the patient was breathing in the alveolar plateau phase. The measured volume of expired air was then injected into a Perkin-Elmer N930-7000 ATD tube packed with Carbotrap 300, and capped. ATD tubes were then desorbed onto the cold trap for 20 min at 320° C.; the cold trap was held at −30° C. and then reapidaly heated to 350° C. and the volatiles liberated were swept onto either one or two 30 m×0.32 mm (10) PLOT GC columns (PEQ) by helium at 2 ml/min to measure both hydrocarbons evolved and also more polar compounds of higher molecular weight. The oven was set at 45° C. for 3-10 minutes and ramped at 14° C. per minute to 200° C. where it was held for 2 minutes.

The various components were identified and quantified by mass spectrometry (Perkin-Elmer Turbo Mass) which was set to monitor mass 43. Butane eluted at 9.62 min, which was confirmed by its mass spectra and an authentic standard gas mixture C1-C6 (Supelco).

Results

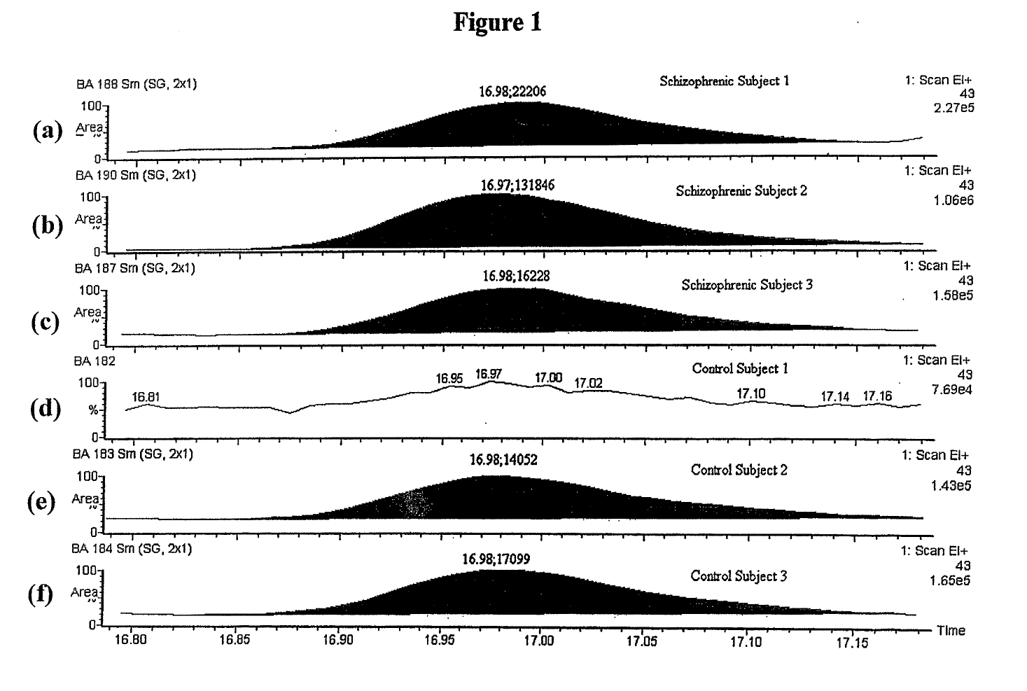

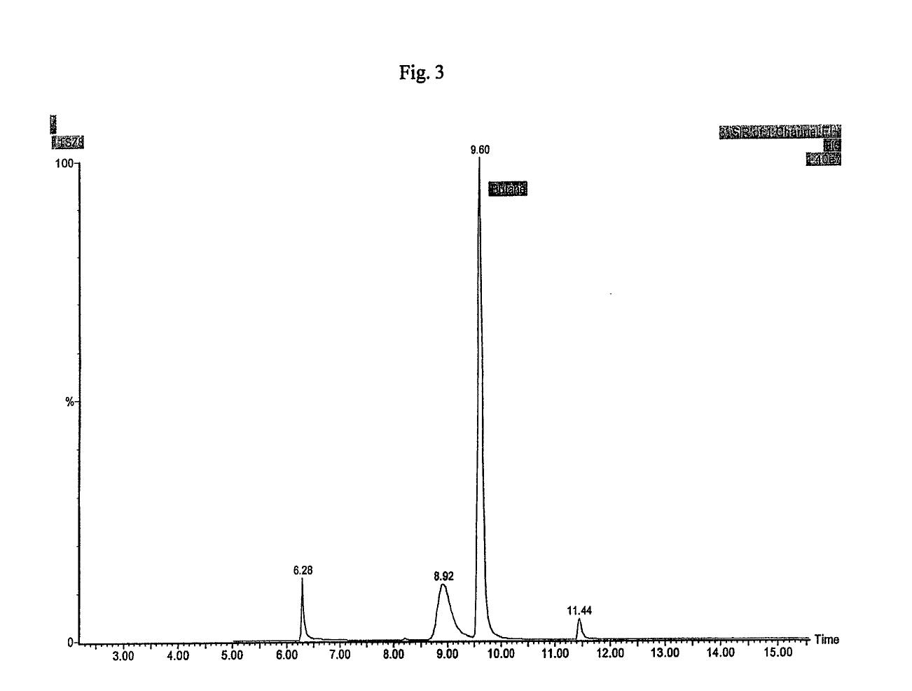

Table 1a gives the area under butane peaks in expired breath from seven known schizophrenics and four normal controls. The peaks are shown in FIG. 1. A typical butane chromatograph is shown in FIG. 3. Table 1b gives the concentration of butane for patients suffering from other conditions compared with 3 control subjects.

TABLE 1a
butane and schizophrenia.

| | AREA OF BUTANE | |
|---|---|---|
| SUBJECT | PEAK (Units) | COMMENTS |
| Schizophrenia | 22206 | Non-smoker |
| patient 1 | | chronically ill |
| | | fairly stable on |
| | | medication |
| Schizophrenia | 131846 | Unmedicated very |
| patient 2 | | ill patient |
| Schizophrenia | 16266 | Smoker who is well |
| patient 3 | | stabilised on |
| | | medication - who |
| | | was ready for |
| | | discharge |
| Schizophrenia | 51348 | Smoker - quite ill |
| patient 4 | | though stable on |
| | | medication |
| Schizophrenia | 56934 | Smoker - quite ill |
| patient 5 | | though stable on |
| | | medication |
| Schizophrenia | 62821 | Non-smoker - |
| patient 6 | | chronically ill |
| | | though stable on |
| | | medication |
| Schizophrenia | 151219 | Smoker chronically |
| patient 7 | | ill but never |
| | | reached stability |
| | | on medication |
| Control subject 1 | 0 | Non-smoker |
| Control subject 2 | 14052 | Smoker |
| Control subject 3 | 17099 | Smoker |
| Control Subject 4 | 15027 | Smoker |

TABLE 1b
butane and other conditions

| | Concentration of | |
|---|---|---|
| Condition | butane ng/L | Comments |
| Huntington's | 1731.40 | Non-smoker |
| chorea (n = 1) | | |
| Dyslexia | 2.00 | Non-smoker |
| (n = 1) | | |
| Alcoholism | 9.08 | Smoker |
| (n = 1) | | |
| Control | 0 | Non-smoker |
| (n = 3) | | |

n = Number of subjects

These results show that the schizophrenics and sufferers of other disorders have more butane in expired breath than normal subjects. The butane concentrations reflect quantitatively how ill the patient is. Smoking apparently increases butane levels. In order to control for the elevation of butane caused by smoking rather than by the presence of schizophrenia the subjects were optionally tested at least 1-3 hours after smoking and it was found (results not shown) that butane levels had decreased in unaffected subjects.

### EXAMPLE 2

### Detection of Ethane

The samples were collected and assayed as described above in example 1. The various components were identified and quantified by mass spectrometry (Perkin-Elmer Turbo Mass) which was set to monitor mass 30. Ethane eluted at 3.65 min, which was confirmed by its mass spectra and an authentic standard gas mixture C1-C6 (Supelco). A standard curve of ethane (0-1.5 ng/L) was run to quantify standards.

Results for Example 2

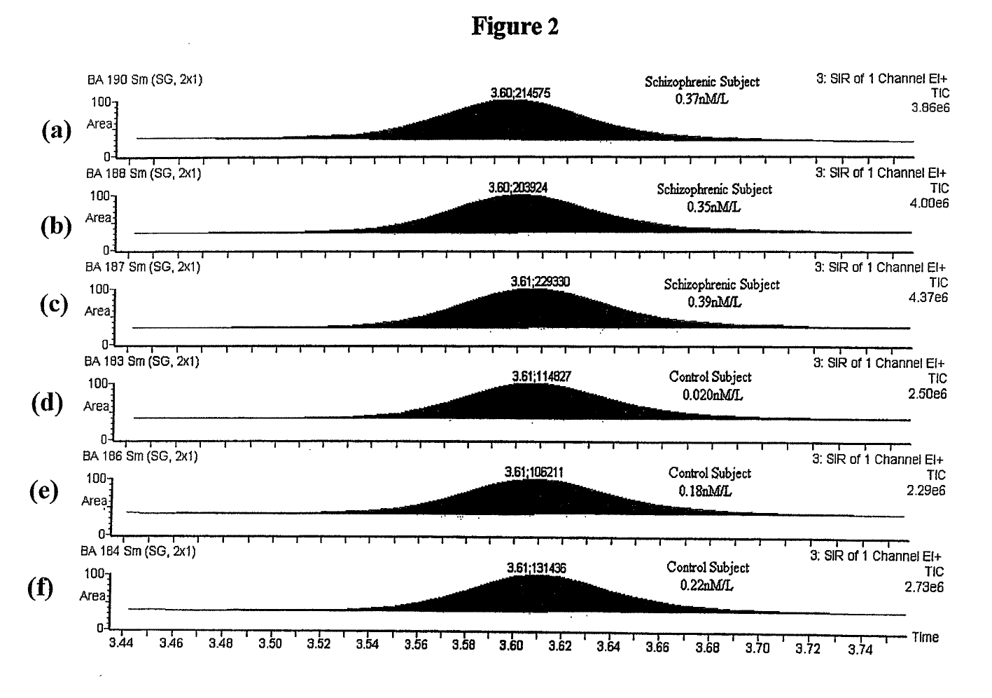

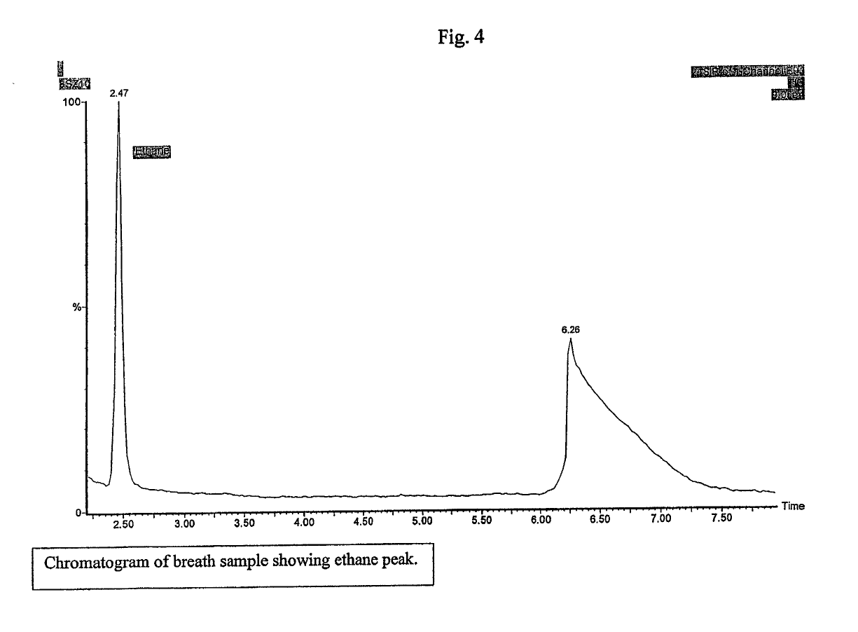

FIG. 2a to 2c show the ethane peak in expired breath from three known schizophrenics and three normal controls. The results are summarised in table 2 below. A typical ethane chromatograph is shown in FIG. 4.

TABLE 2

| | Concentration of | |
|---|---|---|
| | ethane | Mean |
| Schizophrenic 1 | 0.37 nM/L | 0.37 nM/L |
| Schizophrenic 2 | 0.35 nM/L | |
| Schizophrenic 3 | 0.39 nM/L | |
| Normal 1 | 0.20 nM/L | 0.20 nM/L |
| Normal 2 | 0.18 nM/L | |
| Normal 3 | 0.22 nM/L | |

All subjects were smokers. In order to control for the elevation of ethane caused by smoking rather than by the presence of schizophrenia the subjects were optionally tested at least 1 hour after smoking and it was found (results not shown) that ethane levels had decreased in unaffected subjects.

These results show that the schizophrenics have on average almost twice as much ethane in expired breath as normal subjects. The concentration can be determined by measuring the area under the curves of FIG. 2.

The concentration of ethane in the expired breath of patients suffering from other conditions is summarised in table 3 below.

TABLE 3

| | Concentration of | |
|---|---|---|
| Condition | ethane ng/L | Comments |
| Huntington's | 28.15 | Non-smoker |
| chorea (n = 1) | | |
| Dyslexia | 2.23 | Non-smoker |
| (n = 1) | | |
| Alcoholism | 59.67 | Smoker |
| (n = 1) | | |
| Control | 0 | Non-smoker |
| (n = 3) | | |

n = Number of Subjects

Further methods for monitoring ethane in breath are disclosed in Arterbery et al Free Radical Biology and Medicine VOL 17, No 6, page 569-567 1994.

### EXAMPLE 3

End-expired air samples were collected from patients using a Modified Haldane-Preistley tube (125 ml), or a Vacu-sampler partially vacuum (500 mgHg nitrogen) can (MDA Scientific Inc, Park Ridge, Ill., USA), or a GaSampler (Quintron, Milwaukee, Wis.), or patients breath continuously over a period of time via breathing apparatus into a Teflon or Tedlar bag (10-20 litres) whilst breathing in hydrocarbon-free air. The expired air was either aspirated into a rubber free 60 ml plastic syringe (Fortuna Syringe; Aldrich Chemical Co.) or an air-tight glass syringe.

The sample of expired air was then injected into a gas sampling valve, and transferred to a 10 ml sampling loop, attached to gas chromatograph e.g. Hewlet Packard Model 5880A or 5980A, Varian Model 600 or 3400 (Varian Instruments), or a Perkin Elmer Sigma 2000. The sampling loop was flushed with 40 ml of breath and manually pressurised to 800 mmHg with the last 20 ml of the sample by use of a digital manometer (UM2000/200; Netech, Hicksville, N.Y.). The sample was cold-trapped using a cryogenic cold trap before being vaporised on to the column in a discrete band.

Alternatively a breath sample was collected as previously described and then transferred to an automatic thermal desorption tube, sample tube or silanized glass tube packed with Tenax, Chrompak, Carbotrap or Carbotrap/Carboseive 111 (SupelcoUK, Poole Dorset). Tubes were desorbed on the gas chromatograph using a Thermal Desorption Unit (Supelco), ATD 400 (Perkin Elmer) or 4001 TCT (Chrompack UK Ltd, London) with optional cryofocussing on line using liquid nitrogen to cool to −180° C.—desorption 5 min at 300° C. Samples were desorbed at temperatures of between 250° C. to around 350° C. for between 2-15 min and trapped on a cold trap at temperatures below 4° C.

Separation of hydrocarbons (ethane and butane) was carried out on a column e.g. Activated Alumina, Al₂O₃/KCl/PLOT (50 m×0.53 mm, carrier gas flow rate 7.6 ml/min), Carbopack B, Chormosorb 102, GSQ (30 m×0.53 mm, J&W), Porapak T, Poraplot U (30 m×0.32 mm or 10 m×0.53 mm,carrier gas flow rate 3 ml/min, Chrompak), Poraplot Q (10 m×0.53 mm, Chrompack, carrier gas flow 4 ml/min), Porapak N 80/100 (1.5 m, carrier gas flow rate 45 ml/min), Porasil C (15 m×3.18 mm, 80-100 mesh, Alltech, carrier gas flow rate 60 ml/min) or D. The temperature of the gas chromatograph was usually programmed at 40-5° C. for 2-10 min and ramped by 8-15° C./min to a maximum of 180-250° C., and this temperature was held for up to 30 min. The carrier gas was an inert gas e.g. nitrogen, helium or argon. Hydrogen gas was required if a flame ionisation detector was used, and this was usually set at a temperature 240° C.

The various components were identified and quantified by either a flame ionisation detector e.g. Hewlet Packard, Perkin Elmer, Shimadzu GC-8A or a mass spectrometer e.g. Ion Trap detector 800 Finnigan, Perkin Elmer Turbo Mass, Hewlet Packard. Identification of ethane and butane was determined by running a hydrocarbon mixture C1-C6 Paraffins (Scott Specialty Gases, Sigma). A standard curve of ethane and butane was run (0-2 ng/L).

References

Cailleux A, Allain, P. (1993) Free Rad. Res. Comms. 18: 323-327.

Drury J A, Nycyk J A, Cooke R W I. (1997) Free Rad. Biol. Med 22: 895-900.

Euler D E, Dave S J, Guo H. (1996) Clin. Chem. 42:303-308.

Knutson M D, Viteri F E. (1996) Anal. Biochem. 242:129-135.

Mendis S, Sobotak P A, Euler D E. (1994) Clin. Chem. 40:1485-1488.

Springfield J R, Levitt M D. (1994) J. Lipid Res. 35:1497-1504.

Sobotka P A, Brottman M D, Weitz Z, Birnbaum A J, Skosey J L, Zarling E J. (1993) Free Rad. Biol. Med. 14:643-647.

### EXAMPLE 4

Breath samples were collected as previously described. The breath sample was transferred via a pump to activated charcoal housed in ceramic traps (Analyt, Müllheim, Germany). A Microwave thermoinjector (model MW1, Analyt, Müllheim, Germany) which heats up to 600° C. in 10 secs, transferred the volatiles from the breath sample on to the gas chromatograph. Samples were separated by gas chromatography (Hewlett Packard 5980 Series A) using a capillary column CP Sil 8 CB (50 m, Chrompack) and identified by flame ionisation detector or mass spectrometry. The GC temperature was held at 50° C. for 2 min after injection, and subsequently raised to 40° C. by 2° C./min and thereafter increased to 280° C. by 1° C./min for another 5 min.

The samples were analysed for ethane and butane and quantified as previously described.

Reference

Schubert J K, Muller W P E, Benzing A, Geiger K. (1998) 24: 415-421.

### EXAMPLE 5

Breath samples were collected as previously described and transferred to a stainless steel bomb-followed by cryofocussing and vaporization onto a gas chromatograph. Samples were analysed for ethane and butane as previously described.

Reference

Kohlmuller D., Kochen W. Anal. Biochem. (1993) 210:268-276).

### EXAMPLE 6

Expired air was collected into 5 to 10 litre Rislan bags (ATO Emballages S.A., F-93521 St. Denis, Cédex, France) filled to about ⅘ of their capacity. The bag opening was made of glass tubing (3 cm in diameter) which was immediately closed with a Teflon septum after expired air collection. Before analysis, the bags were placed on a hot plate (35° C.) to ensure complete evaporation of hydrocarbons (ethane and butane). After elimination of the water vapour and the CO₂ in a trap containing 5 gm NaOH pellets, the hydrocarbons (ethane and butane) were concentrated on a silica gel column kept at 0° C.

The volume of air aspirated (flow rate 13 ml/min) from the collecting bag through the gas trap maintained at 0° C. was 150 ml. The 3-mm internal diameter U-shaped glass trap was approx. 38 cm long and filled with 1.3 g silica gel (30-60 mesh).

Trap desorption was carried out at 290° C. Hydrocarbon analysis is performed with an Intersmatmodel 131 gas chromatograph fitted with a six-way gas sample valve and equipped with a flame ionisation detector and a stainless steel column (internal diameter 2.2 mm; length 3.2 mm packed with Porasil C (100-150 mesh). A carrier gas such as nitrogen at a flow rate of 13 ml/min. the detector was set at 320° C. The column temperature was programmed as follows: 70° C. for 75sec and ramped at 48° C. /min to 140° C. , and held at this temperature for 3 min.

Identification and quantification was carried out as before by the use of standard curves of standard gases ethane and butane.

Reference

Hotz P., Hoet P., Lauwerys R., Buchet J-P. Clin. Chim. Acta (1987) 163: 303-310.

### EXAMPLE 7

Breath samples were collected as before and were pumped through an ‘electronic nose’ e.g. FOX instrument with 12 metal oxide sensors (Alpha M.O.S., Toullouse, France) (Marcou, et al., 1998) or a similar sensor (Brudzewski and Osowski, 1999). Alternatively the breath samples were pumped through an optical scattering sensor (Guiliana and Jarvis, 1984) or by a microcalometric sensors ( Sommer et al, 1992). The instrument was calibrated with standard hydrocarbon C1-C6 (Scot Speciality Gases, Sigma). Quantification and identification of ethane and butane was carried as previously described.

Reference

Marcou H, Pereira D, Verschuere L, Philips S, Verstraete W. (1998) Water, Air and Soil Pollution 107:423-442.

Brudzewski K, Osowski S. 1999) Sensors and Actuators B 55:38-46.

Guiliani J F, Jarvis N L. (1984) Sensors and Actuators 6:107-112.

Sommer V, Rongen R, Tobias P, Kohl D. (1992) Sensors and Actuators B 6:262-265.

### EXAMPLE 8

Intra-person Variation of butane exhalation.

Samples of expired breath were collected from a schizophrenic patient and a control subject at intervals throughout a period of 3 days, and these samples were analysed for the concentration of butane as described in example 1 above.

Repeated measures of exhaled butane of a patient and a control subject over a period of 3 days are shown in table 4 below. It can be seen that there is a considerable variation over time of the concentration of butane in the patient with schizophrenia compared to that of the control subject. This variation (apart from the elevated levels of butane themselves) gives a further indication that the patient is schizophrenic.

TABLE 4

| | | | Conc |
|---|---|---|---|
| | | | Butane |
| Subject | Day | Time | ng/L |
| Schizophrenic | 1 | 12 noon | 5.23 |
| | | 2.15 pm | 4.84 |
| | | 4.15 pm | 24.38 |
| | 2 | 11.25 am | 8872.65 |
| | | 2 pm | 4.23 |
| | 3 | 10.30 am | 22.15 |
| Control | 1 | 9.30 am | 0 |
| | | 11.45 am | 0 |
| | | 3.30 pm | 0 |
| | | 7.45 pm | 0 |
| | 2 | 4.30 pm | 0 |
| | 3 | 10.30 am | 0 |

While we do not wish to be bound by hypothesis, we can say that this variation may be the result of a dysfunctional control mechanism in the patient with schizophrenia, which may affect the regulation of butane levels although this may not be the primary or only effect of the dysfunction, and that such variation in the butane concentration in expired breath can serve as an even more helpful and specific indicator of the psychiatric condition than looking at snapshots of the butane levels. Other psychiatric conditions could be indicated by the same variation in butane levels in expired breath.

### EXAMPLE 9

Variation of butane levels in other psychiatric conditions.

Samples of expired breath were collected from patients with known schizophrenia and bipolar disorder as described previously, and were compared with control subjects. These samples were analysed for the concentration of butane as described in example 1 above.

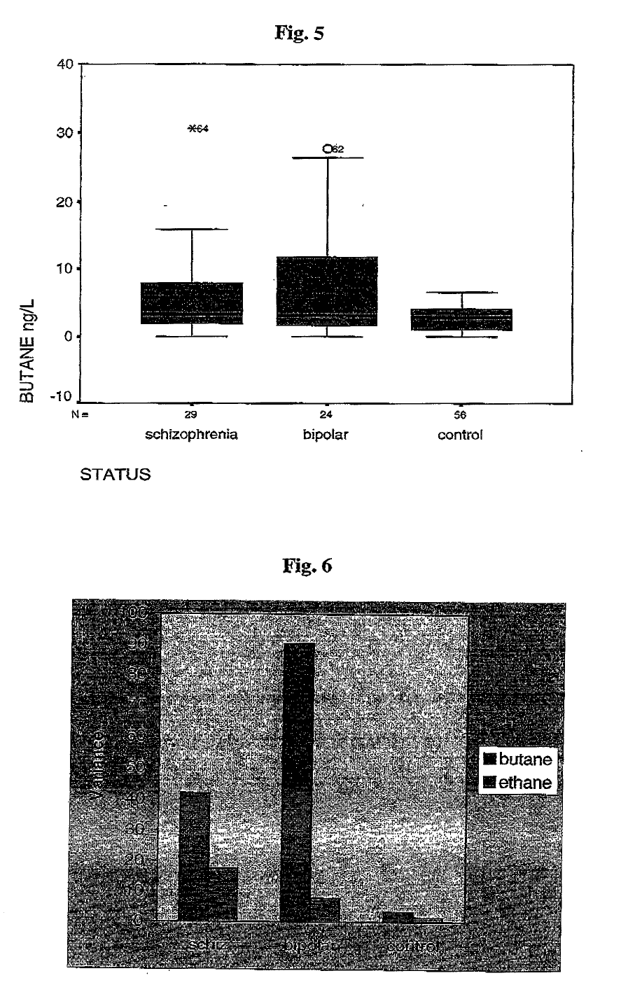

The boxplot shown in FIG. 5 indicates the concentration of butane in the breath of subjects with schizophrenia and bipolar disorder compared to that of the control subjects. The subjects in all groups were both non-smokers and smokers. The maximum value for each group is 6.61 ng/L butane for the controls, 27.76 ng/l for those with bipolar disorder and 30.68 ng/L for those with schizophrenia.

It can be seen from FIG. 5 that subjects with schizophrenia and the bipolar disorder have higher concentrations of butane in their breath compared to that of controls. There were two outliers in the above study. One subject with bipolar disorder had a value >300 ng/L butane in breath and one control subject out of fifty-six had a value >20 ng/L in their breath.

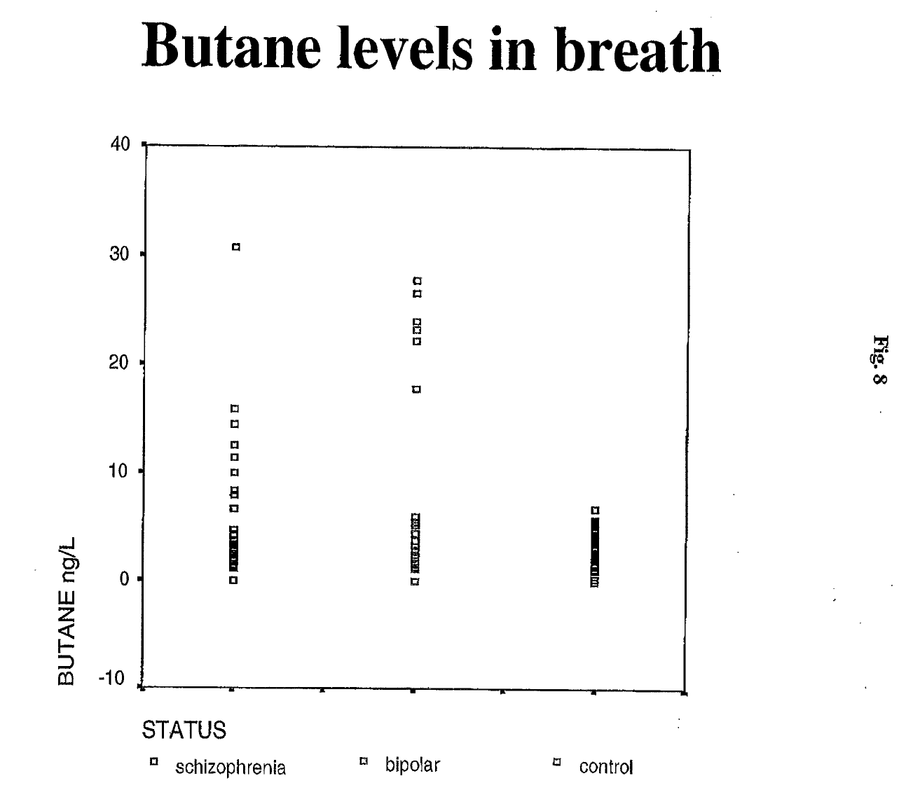

The origins of butane are unclear. However, the FIG. 6 comparison of the variation of butane with ethane shows a greater variation in the levels of butane in the breath of those with schizophrenia and bipolar disorder, and shows that measuring butane is significantly more sensitive than ethane as an indicator of such disorders. The levels of butane in the different conditions of schizophrenia and bipolar disorder from which the variation can also be seen is also shown in FIG. 8. Therefore butane may not be directly related to lipid peroxidation.

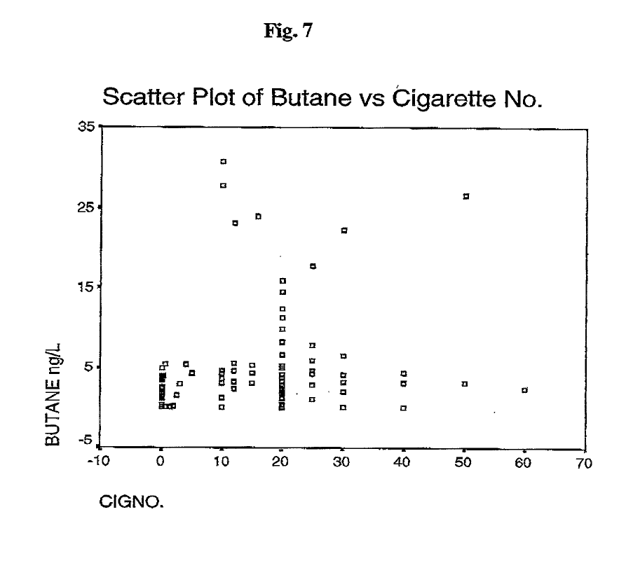

The concentration of butane is not related to the number of cigarettes smoked. As can be seen from the scatter plot shown in FIG. 7, there is no correlation between the number of cigarettes smoked and the amount of butane in breath.

Therefore, it can be seen from these results that the patients with bipolar disorder as well as schizophrenia show an increase in butane concentration in the expired breath, and that the variation in the levels of butane in such patients is also increased significantly over the control levels. Variation in butane levels also appears to be more marked in the case of bipolar disorder than in the case of schizophrenia.

Other diagnostic methods may be used in conjunction with the method of the invention. Therefore, the invention provides for diagnosis or monitoring of the aforementioned conditions, specifically schizophrenia, bipolar disorder, Huntington's chorea, dyslexia and alcoholism, and is achieved by measuring an increase in the concentration of ethane and/or butane in patients' breath, compared to that of normal controls.

Modifications and improvements can be incorporated without departing from the scope of the invention. All references cited are incorporated herein by reference.

1. A method for the assessment of psychiatric or neurological conditions selected from the group consisting of schizophrenia, bipolar disorder, depression, dementia, Huntingdon's chorea, Down's syndrome, Alzheimer's disease, dyspraxia, stroke, head injury and autistic spectrum disorder, the method comprising determining the presence and/or amount of butane in the expired breath of the patient.

2. A method as claimed in claim 1, comprising the additional step of determining the presence and/or amount of ethane in the expired breath of the patient.

3. A method as claimed in claim 1, wherein the amount of butane is measured and correlated to the severity of the condition in a quantitative manner.

4. A method as claimed in claim 1, wherein the butane in expired breath is thermally desorbed onto a matrix before it's presence or amount is determined.

5. A method as claimed in claim 1, wherein the butane is separated from the expired breath by chromatography.

6. A method as claimed in claim 1, wherein the presence or amount of butane determined by mass spectroscopy.

7. A method as claimed in claim 1, wherein the expired breath to be assayed in the method is captured in a breath capture device, and thereafter desorbed onto an absorbent matrix.

8. A method as claimed in claim 1, wherein the butane is analysed by gas chromatography and mass spectrometry.

9. A method as claimed in claim 1, wherein the presence or amount of butane in the expired breath is determined by flame ionisation detection.

10. A method as claimed in claim 1, wherein the presence or amount of butane is determined by analysis selected from the group consisting of infra-red chromatography, fourier transformed infra-red spectroscopy, laser spectroscopy, nuclear magnetic spectroscopy, differential thermal analysis and differential scanning calorimtery.

11. A method as claimed in claim 1, wherein the presence or amount of butane in the expired breath is determined by an electronic device.

12. A method as claimed in claim 1, wherein the butane concentration in expired breath of the patient is measured on several occasions and the variation between the butane levels on each said occasion is determined, and wherein the variation is used as an indicator of the presence of the psychiatric or neurological condition.

13. A method as claimed in claim 12, wherein the variation in the butane levels is used as an indicator of the severity of the psychiatric or neurological condition.
